(19) United States
(12) Patent Application Publication
(10) Pub. No.: US 2018/0071181 A1
(43) Pub. Date: Mar. 15, 2018

(54) COSMETIC COMPOSITIONS COMPRISING A TALC PARTICULATE

(71) Applicant: Imerys Talc Europe, Toulouse (FR)

(72) Inventors: Gilles MELI, Le Fauga (FR); Laure PAGIS, Toulouse (FR)

(21) Appl. No.: 15/558,886

(22) PCT Filed: Mar. 16, 2016
(86) PCT No.: PCT/EP2016/055730

§ 371 Date: Sep. 15, 2017

(30) Foreign Application Priority Data
Mar. 18, 2015 (EP) 15290077.5
Apr. 20, 2015 (EP) 15164298.0
Jan. 11, 2016 (EP) 16305020.6

(51) Int. Cl. A61K 8/25; A61K 8/02; A61Q 1/02

(52) U.S. Cl. CPC A61K 8/25; A61K 8/022; A61K 2800/42; A61K 2800/43; A61Q 1/02

(57) ABSTRACT

Cosmetic compositions comprising a talc particulate which is used in the cosmetic composition as a colour booster, coverage enhancer, opacity enhancer, cohesion enhancer and/or crack prevention additive.

### TECHNICAL FIELD

The present invention is directed to cosmetic compositions comprising a talc particulate which is used in the cosmetic composition as a colour booster, cohesion enhancer, coverage enhance, opacity enhancer, and/or crack prevention additive.

### BACKGROUND OF THE INVENTION

Inorganic particulate materials, such as talc particulate, are commonly used as fillers in cosmetic products, such as pressed powders. There is an ongoing need to develop new cosmetic products having enhanced optical and physical properties.

### SUMMARY OF THE INVENTION

According to a first aspect, the present invention is directed to a cosmetic composition comprising colourant and a talc particulate having a d₅₀ₗₐₛₑᵣ of at least about 5.0 μm, a lamellarity index of at least about 1.0, and wherein the talc particulate comprises less than about 20% aluminium, based on the weight of the talc particulate. In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to a second aspect, the present invention is directed to a cosmetic composition for application to human skin, said cosmetic composition comprising colourant and a colour boosting amount of a talc particulate, wherein the talc particulate boosts the colour of the cosmetic composition (i) such that a lower amount of the cosmetic composition may be applied to any given surface area of the skin without loss of colour, and/or (ii) such that the colour density of the cosmetic composition is boosted compared to the cosmetic composition absent the talc particulate, and/or (iii) a lower amount of colourant may be used without loss of colour density compared to the cosmetic composition absent the talc particulate.

According to a third aspect, the present invention is directed to a cosmetic method for modifying the visual appearance of a facial feature, comprising applying, to the facial feature, a cosmetic composition comprising colourant and a colour boosting amount of a talc particulate.

According to a fourth aspect, the present invention is directed to the use as a colour booster in a cosmetic composition of a talc particulate.

According to a fifth aspect, the present invention is directed to a pressed powder cosmetic comprising a cohesion enhancer, said cohesion enhancer comprising, consisting essentially of, or consisting of, a talc particulate having a d₅₀ₗₐₛₑᵣ of at least about 5.0 μm, and a lamellarity index of at least about 1.0, and wherein the talc particulate comprises less than about 20% aluminium, based on the weight of the talc particulate. In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to a sixth aspect, the present invention is directed to the use as a cohesion enhancer in a pressed powder cosmetic of a talc particulate.

According to a seventh aspect, the present invention is directed to a method for enhancing the cohesion of a powdered cosmetic compact, the method comprising incorporating a talc particulate having a d₅₀ₗₐₛₑᵣ of at least about 5.0 μm and a lamellarity index of at least about 1.0 during manufacture of the powdered cosmetic compact. In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to an eighth aspect, the present invention is directed to a pressed powder cosmetic comprising a crack prevention additive, said crack prevention additive comprising, consisting essentially of, or consisting of, a talc having a d₅₀ₗₐₛₑᵣ of at least about 5.0 μm, and a lamellarity index of at least about 1.0, and wherein the talc particulate comprises less than about 20% aluminium, based on the weight of the talc particulate. In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to a ninth aspect, the present invention is directed to the use as a crack prevention additive in a pressed powder cosmetic of a talc particulate.

According to a tenth aspect, the present invention is directed to a method for preventing cracking of a pressed powder cosmetic, the method comprising incorporating a talc particulate in slurry form in a pressed powder cosmetic compact precursor, and wet processing said precursor to produce a pressed powder cosmetic which is visually free of cracks, optionally wherein (i) the talc particulate has d₅₀ₗₐₛₑᵣ of at least about 5.0 μm, and a lamellarity index of at least about 1.0, and/or (ii) the talc particulate is uncoated, and/or (iii) the pressed powder cosmetic comprises an oil-based binder. In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to an eleventh aspect, the present invention is directed to the use as a colour booster, cohesion enhancer and crack prevention additive in a pressed powder cosmetic of a talc particulate, optionally wherein the talc particulate has d₅₀ₗₐₛₑᵣ of at least about 5.0 μm, and a lamellarity index of at least about 1.0, optionally wherein the talc particulate comprises less than about 20% aluminium, based on the weight of the talc particulate In certain embodiments, the talc particulate has a d₅₀ₗₐₛₑᵣ of at least about 10.0 μm, a lamellarity index of at least about 1.0, and comprises less than about 10% aluminium, based on the weight of the talc particulate.

According to a twelfth aspect, the present invention is directed to a cosmetic composition comprising a coverage and/or opacity enhancer, said coverage and/or opacity enhancer comprising, consisting essentially of, or consisting of, a talc particulate having a d₅₀ₗₐₛₑᵣ of at least about 5.0 μm, a lamellarity index of at least about 1.0, for example, at least about 3.0, and an aluminium content of less than about 20%, based on the weight of the talc particulate.

According to a thirteenth aspect, the present invention is directed to a cosmetic composition for application to human skin, said cosmetic composition a coverage and/or opacity enhancing amount of a talc particulate, wherein the talc particulate enhances the coverage and/or opacity of the cosmetic composition (i) such that a lower amount of the cosmetic composition may be applied to any given surface area of the skin without loss of coverage and/or opacity, and/or (ii) such that the coverage and/or opacity of the cosmetic composition is enhanced compared to the cosmetic composition absent the talc particulate; wherein the talc particulate has a d₅₀ₗₐₛₑᵣ of at least about 5.0 μm, a lamellarity index of at least about 1.0, for example, at least about 3.0, and an aluminium content of less than about 20%, based on the weight of the talc particulate.

According to a fourteenth aspect, the present invention is directed to the use as a coverage and/or opacity enhancer in a cosmetic composition of a talc particulate, wherein the talc particulate has a d₅₀ₗₐₛₑᵣ of at least about 5.0 μm, a lamellarity index of at least about 1.0, for example, at least about 3.0, and an aluminium content of less than about 20%, based on the weight of the talc particulate, optionally wherein the cosmetic composition is a foundation or concealer.

According to a fifteenth aspect, the present invention is directed to the use of a surface treated talc particulate in a cosmetic composition, wherein the talc particulate has a d₅₀ₗₐₛₑᵣ of at least about 5.0 μm, a lamellarity index of at least about 1.0, for example, at least about 3.0, and wherein the talc particulate comprises less than about 20% aluminium, based on the weight of the talc particulate.

According to a sixteenth aspect, the present invention is directed to the use of a surface treated talc particulate in a cosmetic composition to enhance the adhesion (e.g., providing a longer lasting cosmetic effect), water repellence, sebum absorption and/or surface smoothness of the cosmetic composition, for example, when applied to human skin, optionally wherein the talc particulate has: (A) a d₅₀ₗₐₛₑᵣ of at least about 10.0 μm, a lamellarity index of at least about 1.0, for example, at least about 3.0, and wherein the talc particulate comprises less than about 10% aluminium, based on the weight of the talc particulate; or (B) a d₅₀ₗₐₛₑᵣ of at least about 5.0 μm, a lamellarity index of at least about 1.0, for example, at least about 3.0, and wherein the talc particulate comprises less than about 20% aluminium, based on the weight of the talc particulate.

The present invention also extends to all of the aspects one to sixteen wherein reference to talc is replaced by reference to inorganic particulate material.

### BRIEF DESCRIPTION OF THE DRAWINGS

FIG. 1 is a graph showing the fracture resistance of a series of pressed tablets prepared in accordance with Example 2.

FIG. 2 comprises photographs of a series of cosmetic compacts prepared in accordance with Example 3.

### DETAILED DESCRIPTION OF THE INVENTION

It has surprisingly been found that certain inorganic particulate materials, particularly talc particulates, may be incorporated in cosmetic preparations to enhance one or more optical and physical properties thereof, in certain embodiments providing an unexpected balance of properties. For example, incorporation of the inorganic particulate materials described herein, such as a talc particulate, may boost the colour (i.e., increase the colour density) of the cosmetic. Additionally or alternatively, incorporation of the inorganic particulate materials described herein may enhance one or more physical properties of the cosmetic, such as, enhancing cohesion and/or reducing or eliminating cracking of pressed powdered cosmetic compacts. Additionally or alternatively, the incorporation of the inorganic particulate materials described herein may enhance the coverage or opacifying properties of the cosmetic composition, for example, in cosmetic compositions such as concealers, foundations, and powders requiring high or full coverage and homogeneity, including corrective and camouflage products. Additionally or alternatively, incorporation of the inorganic particulate materials described here may enhance the adhesion, water repellence, sebum absorption and/or surface smoothness of the cosmetic composition, for example, when applied to human skin. Enhanced adhesion may provide a longer lasting cosmetic effect, i.e., a long-lasting cosmetic, which is desirable from a user's perspective as it means a cosmetic effect may be maintained for longer periods without or with less need for reapplication of the cosmetic composition. The various properties described above may be determined in accordance with the methods described herein or any other suitable panel or instrumental test.

As used herein, the term “cosmetic composition” means a composition intended to be applied to the human body for beautifying, promoting attractiveness, or altering the appearance without affecting the body's structure or functions. In certain embodiments, the cosmetic composition is a decorative cosmetic.

In certain embodiments, the cosmetic composition is a powder (e.g., pressed or loose), a liquid, a gel, a cream (e.g., a cream emulsion), a dispersion or an anhydrous stick. In certain embodiments, the cosmetic composition is a powder, for example, a pressed powder such as, but not limited to, a powdered cosmetic compact. In certain embodiments, the cosmetic composition is a makeup, for example, a face makeup including, but not limited to, primer, concealer, foundation, blush (also known as rouge or blusher), bronzer, eye shadow, mascara, lipstick, contour powder, face powder (often used to set a foundation), highlighter, eyeliner, or eyebrow applicator (e.g., pencil). Loose powders include body powder, for example, baby powder. In certain embodiments, the make-up is a luminous make-up.

As used herein, the term “lamellarity index” is defined by the following ratio:

$$\frac{{d}_{50 laser} - {d}_{50 sedi}}{{d}_{50 sedi}}$$

in which “d₅₀ₗₐₛₑᵣ” is the value of the mean particle size (d₅₀) obtained by a particle size measurement by wet Malvern laser scattering (standard ISO 13320-1) and “d_{50sedi}” is the value of the median diameter obtained by sedimentation using a sedigraph (standard ISO 13317-3), as described below. Reference may be made to the article by G. Baudet and J. P. Rona, Ind. Min. Mines et Carr. Les techn. June, July 1990, pp 55-61, which shows that this index is correlated to the mean ratio of the largest dimension of the particle to its smallest dimension.

In the sedimentation technique referred to above, particle size properties referred to herein for the talc particulate materials are as measured in a well known manner by sedimentation of the particulate material in a fully dispersed condition in an aqueous medium using a Sedigraph 5100 machine as supplied by Micromeritics Instruments Corporation, Norcross, Ga., USA (www.micromeritics.com), referred to herein as a “Micromeritics Sedigraph 5100 unit”, and based on application of Stokes' Law. Such a machine provides measurements and a plot of the cumulative percentage by weight of particles having a size, referred to in the art as the ‘equivalent spherical diameter’ (e.s.d), less than given e.s.d values. The mean particle size d₅₀ is the value determined in this way of the particle e.s.d at which there are 50% by weight of the particles which have an equivalent spherical diameter less than that d₅₀ value. The d₉₅ value is the value at which 95% by weight of the particles have an esd less than that d₉₅ value. Particle size properties may be determined in accordance with ISO 13317-3, or any method equivalent thereto.

In the Malvern laser light scattering technique referred to above, the size of particles in powders, suspensions and emulsions may be measured using the diffraction of a laser beam, based on an application of Mie theory. Such a machine, for example a Malvern Mastersizer S (as supplied by Malvern Instruments) provides measurements and a plot of the cumulative percentage by volume of particles having a size, referred to in the art as the ‘equivalent spherical diameter’ (e.s.d), less than given e.s.d values. The mean particle size d₅₀ is the value determined in this way of the particle e.s.d at which there are 50% by weight of the particles which have an equivalent spherical diameter less than that d₅₀ value. Particle size properties may be determined in accordance with ISO ISO 13320-1, or any method equivalent thereto. For the avoidance of doubt, the measurement of particle size using laser light scattering is not an equivalent method to the sedimentation method referred to above.

As used herein, “specific surface area (BET)” means the area of the surface of the particles of the talc particulate with respect to unit mass, determined according to the BET method by the quantity of nitrogen adsorbed on the surface of said particles so to as to form a monomolecular layer completely covering said surface (measurement according to the BET method, AFNOR standard X11-621 and 622 or ISO 9277). In certain embodiments, specific surface area is determined in accordance with ISO 9277, or any method equivalent thereto.

### Inorganic Particulate Material

The inorganic particulate material may, for example, be talc, an alkaline earth metal carbonate or sulphate, such as calcium carbonate, magnesium carbonate, dolomite, gypsum, a hydrous kandite clay such as kaolin, halloysite or ball clay, an anhydrous (calcined) kandite clay such as metakaolin or fully calcined kaolin, mica, perlite, bentonite, wollastonite, or diatomaceous earth, or magnesium hydroxide, or aluminium trihydrate, or combinations thereof. Any of the afore-mentioned inorganic particulate materials may be surface untreated or surface treated. For example, the inorganic particulate material may be kaolin surface treated with silicone. For example, the inorganic particulate material may be mica surface treated with silicone.

In certain embodiments, the inorganic particulate material is talc, kaolin or mica.

In certain embodiments, the inorganic particulate material is bentonite or wollastonite.

In certain embodiments, the inorganic particulate material is surface treated.

In certain embodiments, the inorganic particulate material is kaolin treated with silicone.

In certain embodiments, the inorganic particulate material is mica treated with silicone.

In one embodiment, the inorganic particulate material is talc particulate. Hereafter, the invention may tend to be discussed in terms of talc particulate, and in relation to aspects where the talc particulate is processed and/or treated. The invention should not be construed as being limited to such embodiments.

In certain embodiments, the talc particulate has a d₅₀ₗₐₛₑᵣ of at least about 6.0 μm, for example, at least about 7.0 μm, or at least about 8.0 μm, or at least about 9.0 μm. In such embodiments, the talc particulate may have a lamellarity index of at least about 2.0, or at least about 3.0, or at least about 4.0.

In certain embodiments, the talc particulate has a d₅₀ₗₐₛₑᵣ of at least about 10.0 μm and a lamellarity index of at least about 1.0.

In certain embodiments, the talc particulate has a lamellarity index of at least about 1.25, or at least about 1.5, or at least about 1.75, or at least about 2.0, or at least about 2.5, or at least about 3.0, or at least about 3.5, or at least about 4.0, or at least about 4.5, or at least about 5.0, or at least about 5.5, or at least about 6.0, or at least about 6.5, or at least about 7.0. In certain embodiments, the talc particulate has a lamellarity index of from about 1.2 to about 10.0, of from about 1.5 to about 8.0, or from about 1.75 to about 7.5, or from about 2.0 to about 8.0 or from about 3.5 to about 8.0, or from about 5.0 to about 7.5, or from about 6.0 to about 7.5, or from about 7.0 to about 7.5, or from about 3.5 to about 5.5, or from about 3.5 to about 5.0, or from about 4 to about 5.0, or from about 4.0 to about 5.0.

In certain embodiments, the talc particulate has a d₅₀ₗₐₛₑᵣ of from about 10.0 μm to about 50.0 μm, for example, from about 10.0 μm to about 40.0 μm, or from about 10.0 μm to about 35.0 μm, or from about 15.0 μm to about 35.0 μm, or from about 15.0 μm to about 30.0 μm, or from about 20.0 μm to about 30.0 μm, or from about 20.0 μm to about 20.5 μm, or from about 20.0 μm to about 25.0 μm, or from about 15.0 μm to about 25.0 μm, or from about 15.0 μm to about 20.0 μm.

In certain embodiments, the talc particulate has a d₅₀ₗₐₛₑᵣ of from about 5.0 μm to about 50.0 μm, for example, from about 5.0 μm to about 35.0 μm, or from about 5.0 μm to about 25.0 μm, or from about 5.0 μm to about 15.0 μm or from about 7.5 μm to about 12.5 μm.

In certain embodiments, in addition to the d₅₀ₗₐₛₑᵣ and lamellarity index described above, the talc particulate has a d_{50sedi} of at least about 1.0 μm, or at least about 1.5 μm, or at least about 2.0 μm, or at least about 2.5 μm, for example, from about 2.5 μm to about 20.0 μm, or from about 2.5 μm to about 15.0 μm, or from about 2.5 μm to about 12.5 μm, or from about 2.5 μm to about 10.0 μm, or from about 2.5 μm to about 7.5 μm, or from about 2.5 μm to about 5.0 μm, or from about 2.5 μm to about 3.5 μm, or from about 1.0 μm to about 3.0 μm, or from about 1.0 μm to about 2.5 μm, or from about 1.0 μm to about 2.0 μm, or from about 1.5 μm to about 2.5 μm.

In certain embodiments, the talc particulate has a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm.

In certain embodiments, the talc particulate has a lamellarity index of from about 1.0 to about 3.0, for example, from about 1.0 to about 2.5, or from about 1.25 to about 2.0, a a d₅₀ₗₐₛₑᵣ of from about 30.0 μm to about 40.0 μm, and optionally a d_{50sedi} of from about 10.0 μm to about 15.0 μm.

In certain embodiments, the talc particulate has a lamellarity index of from about 4.0 to about 6.0, for example, from about 4.5 to about 5.5, a d₅₀ₗₐₛₑᵣ of from about 15.0 μm to about 25.0 μm, and optionally a d_{50sedi} of from about 3.0 μm to about 4.0 μm.

In certain embodiments, the talc particulate has a lamellarity index of from about 3.0 to about 5.0, for example, from about 4.0 to about 4.5, a d₅₀ₗₐₛₑᵣ of from about 15.0 μm to about 25.0 μm, and optionally a d_{50sedi} of from about 3.0 μm to about 4.0 μm.

In certain embodiments, the talc particulate has a lamellarity index of from about 3.0 to about 5.0, for example, from about 4.0 to about 4.0, a d₅₀ₗₐₛₑᵣ of from about 5.0 μm to about 15.0 μm, and optionally a d_{50sedi} of from about 1.0 μm to about 3.0 μm.

In certain embodiments, the talc particulate has a d₉₅ₗₐₛₑᵣ of less than about 100 μm, for example, less than about 90 μm, or less than about 80 μm, or less than about 75 μm, or less than about 70 μm, or less than about 65 μm, or less than about 60 μm, or less than about 55 μm, or less than about 50 μm.

In certain embodiments, the talc particulate additionally has an aluminium content of less than about 20%, based on the total weight of the talc particulate. The aluminium content is calculated as Al₂O₃ content, as may be determined by X-ray Fluorescence Spectroscopy (XFS). In certain embodiments, the talc particulate has an aluminium content of less than about 15% by weight, or less than about 10% by weight, or less than about 8.0% by weight, or less than about 6.0% by weight, or less than about 5.0% by weight, or less than about 4.0% by weight, or less than about 3.0% by weight, or less than about 2.0% by weight, or less than about 1.5% by weight, or less than about 1.0% by weight, or less than about 0.75% by weight. In certain embodiments, the talc particulate has an aluminium content of at least about 0.10% by weight, for example, at least about 0.20% by weight, or at least about 0.40% by weight.

In certain embodiments, the talc particulate has an aluminium content of from about 5% to about 15%, based on the total weight of the talc particulate, for example, from about 8% to about 12%, or about 10%, or about 11%, or about 12%, or about 13%, or about 14%, based on the total weight of the talc particulate.

In certain embodiments, the talc is derived from a synthetic talc, also known as talcose.

In certain embodiments, the talc is an Italian talc, i.e., derived from a natural talc resource based in Italy. In certain embodiments, the talc is a French talc, i.e., derived from a natural talc resource based in France.

In certain embodiments, the talc particulate has a specific surface area (BET) equal to or greater than about 3 m²/g, for example, equal to or greater than about 8 m²/g, equal to or greater than about 8 m²/g, or equal to or greater than about 15 m²/g, or equal to or greater than about 20 m²/g. In certain embodiments, talc particulate has a specific surface area (BET) of from about 5 m²/g to about 30 m²/g, for example, from about 10 m²/g to about 25 m²/g, or from about 15 m²/g to about 25 m²/g, or from about 20 m²/g to about 25 m²/g. In certain embodiments, talc particulate has a specific surface area (BET) of from about 3 m²/g to about 10 m²/g, for example, from about 3 m²/g to about 8 m²/g, or from about 3 m²/g to about 7 m²/g, or from about 4 m²/g to about 6 m²/g.

In certain embodiments, the talc particulate has:
(i) a specific surface area (BET) of least about 10.0 m²/g for a d_{50sedi} between 2.0-8.0 μm;
(ii) a specific surface area (BET) of least about 5.0 m²/g for a d_{50sedi} between 8.0-15.0 μm;
(iii) a lamellarity index of at least about 3.0 for a d_{50sedi} between 2.0-8.0 μm; and/or
(iv) a lamellarity index of at least about 1.0 for a d_{50sedi} between 8.0-15.0 μm.

In certain embodiments, the talc particulate may be characterised in terms oil absorption. A higher relative oil absorption may enhance one or properties of the cosmetic composition such as, for example, sebum absorption. In certain embodiments, the talc particulate has an oil absorption of at least about 50%, for example, at least about 60%, or at least about 70%. Oil absorption (i.e., amount of oil absorbed per amount of particulate, e.g., ml or g of oil per 100 g of particulate) may be determined by any suitable method, for example, ASTM D1483.

In certain embodiments, the talc particulate is uncoated.

As mentioned above, it has surprisingly been found that talc particulate may be used to favourably enhance one or more optical and/or physical properties of a cosmetic composition comprising colourant. For example, in one embodiment, the talc particulate is used as a colour booster to boost the colour of the cosmetic composition. By “boost the colour” or “colour booster” is meant that the colour density of the cosmetic composition is enhanced by incorporation of the talc particulate, as may be determined by spectrocolorimetery in accordance with ISO 12647. The colour density of the cosmetic composition is boosted compared to the cosmetic composition absent the talc particulate, for example, absent the talc particulate having the characteristics (e.g., the particle size distribution) described herein.

Thus, when applied to human skin, the colour of the cosmetic composition comprising colourant and the colour boosting amount of the talc particulate is boosted, meaning that a lower amount of the cosmetic composition may be applied to any given surface area of the skin without loss of colour and/or a lower amount of colourant may be employed without loss of colour density. Being able to use less cosmetic without loss of colour and/or inclusion of less pigment is economically and environmentally beneficial.

As such, in certain embodiments, there is provided a cosmetic method for modifying the visual appearance of a facial feature, comprising applying, to the facial feature, a cosmetic composition comprising colourant and a colour boosting amount of the talc particulate. The presence of the colour boosting amount of the talc particulate boosts the colour such that (i) a lower amount of the cosmetic composition may be applied to any given surface area of the facial feature without loss of colour and/or (ii) a lower amount of colourant may be used without loss of colour density. In certain embodiments, the assessment is made relative to a comparable cosmetic composition comprising no talc particulate or comprising the same amount of a talc particulate having a d₅₀ₗₐₛₑᵣ of less than 10 μm and/or a lamellarity index of less than 1.0. In certain embodiments, the facial feature is one or more of skin, lips, eyebrow or eyelash.

In certain embodiments, the colour boosting talc particulate has a d₅₀ₗₐₛₑᵣ of at least about 5.0 μm, for example, at least about 10.0 μm and a lamellarity index of at least about 1.0, and optionally an aluminium content of less than about 20%, for example, less than about 10%. In certain embodiments, the colour boosting talc particulate has a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm.

In another embodiment, the talc particulate is used as a cohesion enhancer in a pressed powder cosmetic, e.g., compact, to enhance the cohesion (pressability) of the pressed powder. An increase in cohesion may be determined by measuring the fracture resistance of the pressed powdered cosmetic in tablet form. A suitable method for determining fracture resistance is described in the Examples below. Enhanced cohesion enables process improvement and better handling. In certain embodiments, the talc particulate of the pressed powder cosmetic compact has a d₅₀ₗₐₛₑᵣ of at least about 5.0 μm, for example, at least about 10.0 μm, and a lamellarity index of at least about 1.0, and the increase in cohesion of the pressed powder cosmetic compact is measured relative to a comparable powdered cosmetic compact comprising the same amount of a talc particulate which does not have the combination of a d₅₀ₗₐₛₑᵣ of at least about 5.0 μm, for example, at least about 10.0 μm, and a lamellarity index of at least about 1.0. In a related embodiment, there is provided a method for enhancing the cohesion of a pressed powder cosmetic compact, the method comprising incorporating a talc particulate having a d₅₀ₗₐₛₑᵣ of at least about 10.0 μm and a lamellarity index of at least about 1.0 during manufacture of the pressed powder cosmetic compact.

In certain embodiments, the cohesion enhancing talc particulate has a d₅₀ₗₐₛₑᵣ of at least about 5.0 μm, for example, at least about 10.0 μm and a lamellarity index of at least about 1.0, and optionally an aluminium content of less than about 20%, for example, less than about 10%. In certain embodiments, the cohesion enhancing talc particulate has a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm.

In another embodiment, the talc particulate is used, in slurry form, as a crack prevention additive in a pressed powder cosmetic compact, manufactured by a wet processing method, to reduce or eliminate cracking (i.e., visually discernible cracks). Cracking can be a problem in pressed powder cosmetics prepared by wet processing methods. Cracks can cause handling and storage problems and are also aesthetically unpleasing to the user. The crack prevention additive may comprise, consist essentially of, or consist of, a talc particulate material having a d₅₀ₗₐₛₑᵣ of at least about 5.0 μm, for example, at least about 10.0 μm and a lamellarity index of at least about 1.0, and optionally an aluminium content of less than about 20%, for example, less than about 10%. In certain embodiments, the talc particulate for use as a crack prevention additive has a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm. In certain embodiments, the talc particulate for use as a crack prevention additive has a lamellarity index of from about 4.0 to about 6.0, for example, from about 4.5 to about 5.5, a d₅₀ₗₐₛₑᵣ of from about 15.0 μm to about 25.0 μm, and optionally a d_{50sedi} of from about 3.0 μm to about 4.0 μm.

In a related embodiment, there is provided a method for preventing cracking of a powdered cosmetic compact, the method comprising incorporating a talc particulate in slurry form in a powdered cosmetic compact precursor, and wet processing said precursor to produce a powdered cosmetic compact which is visually free of cracks, wherein: (i) the talc particulate has a d₅₀ₗₐₛₑᵣ of at least about 5.0 μm, for example, at least about 10.0 μm, and a lamellarity index of at least about 1.0, and optionally an aluminium content of less than about 20%, for example, less than about 10%; and/or (ii) the talc particulate is uncoated, and/or (iii) the powdered cosmetic compact comprises an oil- or wax-based binder. In certain embodiments, the talc particulate has a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm. In certain embodiments, the talc particulate has a lamellarity index of from about 4.0 to about 6.0, for example, from about 4.5 to about 5.5, a d₅₀ₗₐₛₑᵣ of from about 15.0 μm to about 25.0 μm, and optionally a d_{50sedi} of from about 3.0 μm to about 4.0 μm.

In certain embodiments, the talc particulate has a lamellarity index of from about 3.5 to about 5.0, for example, from about 4.0 to about 5.0, a d₅₀ₗₐₛₑᵣ of from about 8.0 μm to about 12.0 μm, and optionally a d_{50sedi} of from about 1.5 μm to about 3.0 μm, and optionally an aluminium content of from about 10% to about 15%, based on the total weight of the talc particulate.

As mentioned above, it has surprisingly been found that talc particulate may be used to favourably enhance the coverage and/or opacity properties of cosmetic composition. For example, in one embodiment, the talc particulate is used as a coverage and/or opacity enhancer to enhance the coverage and/or opacity of the cosmetic composition. The coverage and/or opacity of the cosmetic composition may be enhanced compared to the cosmetic composition absent the talc particulate, for example, absent the talc particulate having the characteristics (e.g., the particle size distribution) described herein.

Thus, when applied to human skin, the coverage and/or opacity of the cosmetic composition is enhanced, meaning, for example, that a lower amount of the cosmetic composition may be applied to any given surface area of the skin without loss of coverage and/or opacity. Being able to use less cosmetic without loss of coverage and/or opacity, and/or inclusion of less pigment, is economically and environmentally beneficial.

As such, in certain embodiments, there is provided a cosmetic composition, for example, a loose or pressed powder, a liquid, a gel, a cream (e.g., a cream emulsion), a dispersion, or a an anhydrous stick, comprising a coverage and/or opacity enhancer, said coverage and/or opacity enhancer comprising, consisting essentially of, or consisting of, a talc particulate having a d₅₀ₗₐₛₑᵣ of at least about 5.0 μm, a lamellarity index of at least about 1.0, and comprises less than about 20% aluminium, based on the weight of the talc particulate, for example, less than about 15% aluminium.

In certain embodiments, there is provided a cosmetic composition for application to human skin, said cosmetic composition a coverage and/or opacity enhancing amount of a talc particulate, wherein the talc particulate enhances the coverage and/or opacity of the cosmetic composition (i) such that a lower amount of the cosmetic composition may be applied to any given surface area of the skin without loss of coverage and/or opacity, and/or (ii) such that the coverage and/or opacity of the cosmetic composition is enhanced compared to the cosmetic composition absent the talc particulate; wherein the talc particulate has a d₅₀ₗₐₛₑᵣ of at least about 5.0 μm, a lamellarity index of at least about 1.0, and comprises less than about 20% aluminium, based on the weight of the talc particulate, for example, less than about 15% aluminium.

In certain embodiment, the composition is a foundation or concealer.

In certain embodiments, the talc particulate is used as a coverage and/or opacity enhancer in a cosmetic composition of a talc particulate. The talc particulate may have a d₅₀ₗₐₛₑᵣ of at least about 5.0 μm, and a lamellarity index of at least about 1.0. The cosmetic composition may be a foundation or concealer.

In certain embodiments, the coverage and/or opacity enhancer is present in amount sufficient to provide: (i) full coverage when applied to human skin, and/or (ii) an opacity of at least about 70%.

Coverage may be assessed in accordance with the following coverage scale:

‘Sheer coverage’—sheer coverage is the most transparent; it will not hide imperfections on the skin but will illuminate the skin.

‘Light coverage’—light coverage covers unevenness and slight blotchiness, but is not opaque enough to cover freckles.

‘Medium coverage’—medium coverage covers freckles, discolorations, blotchiness and red marks.

‘Full coverage’—full coverage is very opaque, and covers birthmark, hyperpigmentation and scar.

Opacity may be determined in accordance with any suitable method, including the method below in the Examples section below. In certain embodiments a sufficient amount of the talc particulate is used in the cosmetic composition to provide an opacity of at least about 65%, or at least about 70%, or at least about 75%, or at least about 80%, or at least about 81%, or at least about 82%, or at least about 83%, or at least about 84%, or at least about 85%. In certain embodiments, the amount of talc particulate used to provide an opacity of at least about 65% is at least about 50 wt. %, based on the total weight of the composition. In certain embodiments, the amount of talc particulate is from about 60 wt. % to about 99 wt. %, based on the total weight of the composition, for example, from about 65 wt. % to about 95 wt. %, or from about 70 wt. % to about 90 wt. %, or from about 75 wt. % to about 90 wt. %, or from about 75 wt. % to about 85 wt. %, or from about 80 wt. % to about 85 wt. %. In certain embodiments, the amount of talc particulate used in the composition to provide an opacity of at least about 80% is at least about 75 wt. %, based on the total weight of the cosmetic composition, for example, at least about 80 wt. %, based on the total weight of the cosmetic composition.

In certain embodiments, ‘full coverage’ equates to an opacity of at least about 65%, for example, at least about 70%, or at least about 75%, or at least about 80%.

In certain embodiments, the cosmetic composition comprising the talc particulate as opacity enhancer has an opacity of at least about 65%, or at least about 70%, or at least about 75%, or at least about 80%, or at least about 81%, or at least about 82%, or at least about 83%, or at least about 84%, or at least about 85%. In certain embodiments, the cosmetic composition has an opacity of no greater than about 99%, or no greater than about 95%, or no greater than about 90%.

In certain embodiments, for example, embodiments in which the talc particulate is used to enhance, increase or improve coverage or opacity of the cosmetic composition, the talc particulate may have an aluminium content of greater than about 5%, or greater than about 10%, for example, no greater than about 20%, as may be determined in accordance with the method described above.

In certain embodiments, for example, embodiments in which the talc particulate is used to increase or improve coverage or opacity of the cosmetic composition, at least a portion of the talc particulate is a chloritic talc.

In certain embodiments, the talc particulate is used as both a colour booster and cohesion enhancer in a pressed powder cosmetic compact. In certain embodiments, the talc particulate is used as both a colour booster and crack prevention additive in a pressed powder cosmetic compact. In certain embodiments, the talc particulate is used as both a cohesion enhancer and a crack prevention additive in a pressed powder cosmetic compact. In certain embodiments, the talc particulate is used as a colour booster, cohesion enhancer and crack prevention additive in a pressed powder cosmetic compact. In certain embodiments, the talc particulate is used as a colour booster and coverage and/or opacity enhancer. In certain embodiments, the talc particulate is used as a coverage and/or opacity enhancer and crack prevention additive. In certain embodiments, the talc particulate is used as a coverage and/or opacity enhancer and cohesion enhancer. In certain embodiments, the talc particulate is used as a coverage and/or opacity enhancer, a crack prevention additive and a cohesion enhancer. In certain embodiments, the talc particulate is used as a colour booster, a coverage and/or opacity enhancer, a crack prevention additive and a cohesion enhancer. Thus, in certain embodiments, the talc particulate provides an advantageous and unexpected balance of properties. In such embodiments, the talc particulate may have a d₅₀ₗₐₛₑᵣ of at least about 10.0 μm and a lamellarity index of at least about 1.0, and optionally an aluminium content of less than about 10%. In such embodiments, talc particulate may have a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm. In such embodiments, the talc particulate may have a d₅₀ₗₐₛₑᵣ of at least about 5.0 μm and a lamellarity index of at least about 1.0, for example, at least about 3.0, for example, from about 4.0 to about 5.0, and optionally a d_{50sedi} of from about 1.5 μm to about 3.5 μm. In such embodiments, the talc particulate may have a d₅₀ₗₐₛₑᵣ of at least about 15.0 μm and a lamellarity index of at least about 1.0, for example, at least about 3.0, for example, from about 4.0 to about 5.0, and optionally a d_{50sedi} of from about 2 μm to about 5 μm. The wet processing method comprises preparing an aqueous slurry of the talc particulate and any other components of the pressed powder cosmetic compact, for example, colourant (such as mineral pigment) and binder, and then de-watering the slurry, e.g., by drying at a suitably elevated temperature (e.g., above about 70° C.), and then pressing to form a pressed powder cosmetic compact.

In certain embodiments, the incorporation of the talc particulate enhances the homogeneity (i.e., pigment dispersion and homogeneity of color) of the cosmetic composition following application to human skin (or lips if in a lipstick or lipliner). Homogoneity may be determined using any suitable spectroscopic or optical method, including visual assessment in a controlled panel test. The homogeneity may be enhanced compared to a cosmetic composition absent the talc particulate or compared to a cosmetic composition which comprises the same amount of a talc particulate which does not possess either: (i) a d₅₀ₗₐₛₑᵣ of at least about 10.0 μm and a lamellarity index of at least about 1.0, and an aluminium content of less than about 10%; or (ii) a d₅₀ₗₐₛₑᵣ of at least about 5.0 μm, a lamellarity index of at least about 1.0, for example, at least about 3.0, for example, from about 4.0 to about 5.0, an aluminium content of less than about 20%, for example, less than about 15%, and optionally a d_{50sedi} of from about 1.5 μm to about 3.5 μm. Additionally or alternatively, the talc particulate may impart an unctuous and/or creamier skin-feel compared to a cosmetic composition absent the talc particulate or compared to a cosmetic composition which comprises the same amount of a talc particulate which does not possess either: (i) a d₅₀ₗₐₛₑᵣ of at least about 10.0 μm and a lamellarity index of at least about 1.0, and an aluminium content of less than about 10%; or (ii) a d₅₀ₗₐₛₑᵣ of at least about 5.0 μm, a lamellarity index of at least about 1.0, for example, at least about 3.0, for example, from about 4.0 to about 5.0, an aluminium content of less than about 20%, for example, less than about 15%, and optionally a d_{50sedi} of from about 1.5 μm to about 3.5 μm. These characteristics may be determined using a suitably controlled panel test.

In certain embodiments, the cosmetic composition comprises from about 1% by weight to about 95% by weight of talc particulate (i.e., based on the total weight of the cosmetic composition) for example, from about 20% to about 90% by weight, or from about 30% to about 90% by weight, or from about 40% to about 90% by weight, or from about 50% to about 90% by weight, or from about 60% to about 90% by weight, or from about 65% to about 85% by weight, or from about 70% to about 85% by weight, or from about 75% to about 85% by weight, or up to about 95% by weight talc particulate, or up to about 90% by weight talc particulate, or up to about 85% by weight talc particulate, or up to about 80% by weight talc particulate.

In certain embodiments, the cosmetic composition comprises a combination of a higher grade talc particulate and a lower grade talc particulate. For example, the higher grade talc particulate may have a d₅₀ₗₐₛₑᵣ of at least about 10.0 μm, a lamellarity index of at least about 1.0 and optionally an aluminium content of less than about 10%, and further embodiments thereof in accordance with the talc particulate described above, and the lower grade talc particulate may have a coarser particle size distribution and/or a lamellarity index of less than 1.0 (or less than 0.75, or less than about 0.5, or less than about 0.25) and/or an aluminium content which is higher than the higher grade talc particulate, for example, an aluminium content of equal to or greater than about 10%, or equal to or greater than 7.5%, or equal to or greater than about 5%. A higher grade talc particulate may require a higher degree of engineering and, thus, may be more expensive to produce compared to a lower grade talc particulate.

The talc particulate may be obtained from a natural source by grinding. Natural talc particulate is typically obtained by crushing and then grinding a mineral source of talc, which may be followed by a particle size classification step, in order to obtain a product having a desired particle size distribution. The particulate solid material may be ground autogenously, i.e. by attrition between the particles of the solid material themselves, or, alternatively, in the presence of a particulate grinding medium comprising particles of a different material from the talc particulate to be ground. These processes may be carried out with or without the presence of a dispersant and biocides, which may be added at any stage of the process.

The talc particulate may be prepared using techniques well known to a person of skill in the art, for example, techniques selected from comminution (e.g., crushing, grinding, milling), classification (e.g., hydrodynamic selection, screening and/or sieving) and drying.

In certain embodiments, the talc particulate is prepared by a process according to that described in U.S. Pat. No. 6,348,536, the entire contents of which are hereby incorporated by reference. More particularly, the talc particulate may be prepared by a process comprising:
(a) talc with a predetermined initial particle size is suspended in a liquid,
(b) the suspension is subjected to a delamination operation adapted so as to produce a separation of the leaves of the particles and so as to obtain a particle size less than the initial particle size,
(c) optionally subjecting the suspension to a selection as to eliminate particles with a size greater than a predetermined size,
(d) drying the suspension, and
(e) optionally treating the particles so as to limit the creation of strong bonds between them.

The starting talc is typically chosen having an initial particle size which is greater than the desired particle size. In certain embodiments, the starting talc is suspended in water in the presence of a dispersing agent such that the weight of dry matter based on the total weight of the suspension is from about 10% to about 60%. The suspension is typically homogenous. The grinding operation during delamination is, in certain embodiments, carried out as to obtain a d₅₀ₗₐₛₑᵣ of from about 10 μm to about 50 μm. The selection step may comprise hydrodynamic selection, which may be carried out in a turbine selector or in a hydrocyclone or in a centrifuge with an endless extraction screw. The suspension is advantageously dried in such a way as to reach a residual liquid level below 1%.

In certain embodiments, the talc particulate is prepared by a process comprising:
(a) delaminating a liquid suspension of a relatively coarse talc particulate having an initial particle size with a d₅₀ₗₐₛₑᵣ which is greater than a desired d₅₀ₗₐₛₑᵣ (e.g., greater than a desired d₅₀ₗₐₛₑᵣ of at least about 5 μm, or from about 10 μm to about 50 um, or from about 10 μm to about 35 μm), to obtain a talc particulate having a particle size less than the initial particle size;
(b) at least partially drying the suspension thereby obtaining a talc particulate having the desired d₅₀ₗₐₛₑᵣ and optionally a desired lamellarity index.

In certain embodiments, the desired d₅₀ₗₐₛₑᵣ is at least about 10 μm, and the desired lamellarity index is at least about 1.0.

In certain embodiments, the desired d₅₀ₗₐₛₑᵣ is at least about 5 μm, and the desired lamellarity index is at least about 1.0, for example, at least about 3.0.

In certain embodiments, the inorganic particulate, for example, talc particulate is not chemically treated during processing to obtain the desired particle size and lamellarity.

In certain embodiments, the cosmetic composition comprises colourant and/or binder and/or cosmetically acceptable base in addition to the inorganic particulate material. In certain embodiments, the binder, when present, may be a constituent of the cosmetically acceptable base. In certain embodiments, the cosmetic composition comprises colourant and binder, in addition to the talc particulate.

The colourant (i.e., a component which imparts colour) may be an organic colourant and/or an inorganic colourant. Colourants for cosmetics are many and various. A list of colorant agents permitted for use in cosmetic products is provided in Annex IV to the Cosmetics Directive 76/768/EEC. Organic colourants include dyes and the like. Examples of organic colourants include species characterized in one of the following groups: indigoid, xanthenes, azo, nitro, triphentlmehtnae, quinoline and anthraquinone. Inorganic colourants include pigments, such as mineral pigments. In certain embodiments, the colourant is a mineral pigments, for example, one or more of zinc oxide, titanium dioxide, iron oxide (black, red, orange, yellow and/or brown), tin oxide, chrome oxide, ultramarine (blue, pink and/or violet), manganese violet (ammonium manganese (III) pyrophosphate), Prussian blue (ferric ferrocynanide) and mica. In certain embodiments, the colourant is a pigment. In certain embodiments, the colourant is a mineral pigment. The colourant, for example, the mineral pigment or combinations thereof, may be selected depending on the desired colour for the cosmetic. In certain embodiments, the colourant may be nacre or a derivative thereof, providing a desirable pearlescence (also known as luster) and/or brilliance. In such embodiments, the incorporation of the inorganic particulate, for example, talc particulate, may function as a nacre-booster, serving either to enhance the pearlescence and/or brilliance of the cosmetic composition in the presence of nacre, or enable a lower amount of nacre to be used without loss of pearlescence and/or brilliance.

The colourant may constitute up to about 40% by be weight of the cosmetic composition, for example, up to about 35% by weight, or up to about 30% by weight, or up to about 35% by weight, or up to about 20% by weight of the cosmetic composition, for example, from about 0.001% to about 20% by weight, or from about 0.01% to about 20% by weight, or from about 0.1% to about 20% by weight, or from about 1.0% to about 20% by weight, or from about 1.0% to about 15% by weight, or from about 2.0% to about 15% by weight, or from about 5.0% to about 15% by weight, or from about 7.5% by weight to about 12.5% by weight of the cosmetic composition.

In certain embodiments, the cosmetic composition is an eye-shadow, for example, a highly coloured eye-shadow. In certain embodiments, the cosmetic composition is an eyeshadow and comprises at least about 30% by weight of talc particulate and at least about 10% by weight of colourant, for example, at least about 35% by weight talc particulate and at least about 20% by weight colourant, or at least about 40% by weight talc particulate and at least about 25% by weight colourant. In such embodiments, the cosmetic composition, i.e., eye-shadow may comprises no more than about 50% by weight talc particulate, for example, no more than about 45% by weight talc particulate, and no more than about 40% by weight colourant, for example, no more than about 35% by weight colourant, or no more than about 30% by weight colourant.

In certain embodiments, the cosmetic composition is a blush and comprises from about 60% by weight to about 90% by weight talc particulate, for example, from about 70% by weight to about 85% by weight talc particulate, or from about 75% by weight to about 85% by weight talc particulate, or from about 75% by weight to about 80% by weight talc particulate.

In certain embodiments, the cosmetic composition is a foundation and comprises from at least about 70% by weight talc particulate, for example, at least about 80% by weight talc particulate, or at least about 90% by weight talc particulate. In such embodiments, the cosmetic composition, for example, foundation, may comprise from about 0.1% by weight to about 10% by weight colourant, for example, from about 0.5% by weight to about 7% by weight colourant, or from about 1.0% by weight to about 5% by weight colourant, or at least about 2% by weight colourant, or at least about 3% by weight colourant.

When present, the cosmetically acceptable base may be any base suitable for the intended purpose. In certain embodiments, the base is an oil and/or wax containing material. The base and, thus, the cosmetic composition, may comprise other components such as humectants, preservative, emollient, fragrance and antioxidant. The binder, when present, may be a solid or liquid binder. In certain embodiments, the binder is a liquid binder, for example, on oil-based binder. In certain embodiments, the liquid binder is a fatty acid or ester or salt thereof, or a combination of fatty acids and/or ester and/or salts thereof. In certain embodiments, the fatty acid or ester or salt thereof, or combinations thereof, is derived from vegetable oil, for example, coconut oil, palm oil, palm kernel oil soybean oil, corn oil, rapeseed oil, and the like. In certain embodiments, the binder is cocoate ester, for example, isoamyl cocoate. Other binders include silicone, sorbitan, calcium phosphate, bentonite, natural guns, rosin and cellulose.

Suitable binder materials include polyhydric alcohol, hyaluronic acid and its salts, an amino acid and its salts, chondroitin sulfuric acid and its salts, lactic acid and its salts, pyroglutamic acid and its salts, uric acid and its salts, and mixtures thereof. Polyhydric alcohols include glycerin, diglycerin, triglycerin, ethylene glycol, diethylene glycol, propylene glycol, dipropylene glycol, hexylene glycol, 1,3-butylene glycol, 1,4-butylene glycol, glucose, maltose, sucrose, xylitose, sorbitol, maltitol, malbit, panthenol, hyaluronic acid and its salts, and mixtures thereof.

Further non-limiting examples of suitable binder materials are polyglycerin fatty acid esters, propylene glycol fatty acid esters, glycerin fatty acid esters, sorbitan fatty acid esters, sugar fatty acid esters, polyoxyethylene sorbitan fatty acid esters, polyoxyethylene sorbit fatty acid esters, polyethylene glycol fatty acid esters, polyoxyethylene castor oils, polyoxyethylene hardened castor oils, polyoxyethylene alkyl ethers, polyoxyethylene phytosterols, polyoxyethylene polyoxypropylene alkyl ethers, polyoxyethylene alkyl phenyl ethers, polyoxyethylene lanolins, polyoxyethylene lanolin alcohols, polyoxyethylene beeswax derivatives, polyoxyethylene fatty acid amides, and polyether silicone derivatives.

The fatty acids making the esters above can be saturated or unsaturated, straight or branched, and include those of natural origin having about 16-18 carbons. Non-limiting examples include triglyceryl beeswax, triglyceryl cetyl ether, tetraglyceryl cocoate, triglyceryl decyltetradecanol, diglyceryl diisostearate, triglyceryl diisostearate, decaglyceryl diisostearate, diglyceryl dioleate, triglyceryl dioleate, hexaglyceryl dioleate, decaglyceryl dioleate, triglyceryl distearate, hexaglyceryl distearate, decaglyceryl distearate, decaglyceryl trioleate, decaglyceryl heptaoleate, decaglyceryl heptastearate, hexaglyceryl hexaoleate, diglyceryl isostearate, tetraglyceryl isostearate, hexaglyceryl monoisostearate, diglyceryl lanolin alcohol ether, tetraglyceryl lauryl ether, diglyceryl oleate, triglyceryl oleate, tetraglyceryl oleate, hexaglyceryl oleate, diglyceryl oleyl ether, tetraglyceryl oleyl ether, diglyceryl sesquiisostearate, and diglyceryl sesquioleate and mixtures thereof.

Also suitable as binder materials are liquid paraffin, squalane, liquid petrolatum, mineral oil, and liquid polybutene.

Also suitable are natural oils which are typically a mixture of saturated and unsaturated fatty acid. Non-limiting examples of natural oil derived from plants include almond oil, olive oil, sesame oil, safflower oil, avocado oil, cottonseed oil, jojoba oil, castor bean oil, castor oil, rapeseed oil, soybean oil, palm kernel oil, coconut oil, hydrogenated vegetable oil, and cocoa butter. Non-limiting examples of natural oil derived from animal sources include mink oil and egg yolk oil.

Non-limiting examples of fatty alcohol which may be employed as binder are isostearyl alcohol, lanolin alcohol, oleyl alcohol, hexadecyl alcohol, octyldodecanol alcohol, linoleyl alcohol, linolenyl alcohol, and arachidyl alcohol.

Fatty acid can be natural or synthetic, saturated, unsaturated, linear, or branched. Non-limiting examples of fatty acid are adipic, caprylic, capric, isostearic, linoleic, ricinoleic, oleic, elaidic and erucic acid.

Non-limiting examples of fatty acid ester are cetyl ricinoleate, cetyl oleate, cetyl octanoate, cetyl acetate, glyceryl trioctanoate, isopropyl lanolate, isopropyl linoleate, isopropyl myristate, isopropyl palmitate, isopropyl oleate, isopropyl stearate, ethyl lactate, ethyl glutamate, ethyl laurate, ethyl linoleate, ethyl methacrylate, ethyl myristate, ethyl palmitate, diisopropyl adipate, octyl dodecyl myristate, octyl palmitate, octyl isopelargonate, octyl dodecyl lactate, tridecyl isononanoate, isotridecyl isononanoate, hexadecyl stearate, oleyl oleate, isononyl isononanoate, isostearyl myristate, dipenta-erythrytol ester, neopentyl glycol dioctanoate, and di(capryl/capric acid) propylene glycol and mixtures thereof. Other suitable esters include triglycerides such as caprylic triglycerides, capric triglyceride, isostearic triglyceride, adipic triglyceride and cholesterol derivatives such as cholesteryl oleate.

Non-volatile, straight, and branched silicone oil such as dimethicone and phenyl dimethicone is also useful.

The binder may constitute up to about 80% by weight of the cosmetic composition, for example, from about 1% to about 70% by weight, or from about 1% to about 69% by weight, or from about 1% to about 50% by weight, or from about 1% to about 40% by weight, or from about 1 to about 30% by weight, or from about 1 to about 20% by weight, or from about 2.5% to about 15% by weight, or from about 5% to about 15% by weight, or from about 7.5% to about 12.5% by weight of the cosmetic composition.

In certain embodiments, the cosmetic composition comprises up to about 95% by weight talc particulate, from about 1.0% to about 20% by weight colourant (e.g., mineral pigment) and from about 1.0% to about 20% by weight binder. In such embodiments, the cosmetic composition may further comprise suitable amounts of one or more of humectants, preservative, emollient, fragrance and antioxidant, for example, up to about 10% by weight so such components, based on the total weight of the composition, or up to about 5% by weight of such components, or from about 0.001% to about 2.5% by weight of such components. In certain embodiments, the cosmetic composition is free of components other than the talc particulate, colourant and binder. The cosmetic composition may be prepared by any suitable or conventional method well known to those skilled in the art. Such methods generally comprise combining the components of the cosmetic in a liquid, slurry or solid form, mixing the components, optionally milling the mixture of components, and then forming the cosmetic composition therefrom. The components may be brought together in a blender or other mixing apparatus under conditions of suitably low shear so as to preserve the inherent properties of the particulate material. Forming may comprise drying and/or pressing, depending on the nature of the method of manufacture and the final form of the cosmetic.

In certain embodiments, the inorganic particulate, for example, talc particulate is surface treated. The surface treatment may serve to modify a property of the talc particulate and/or the cosmetic composition in which it is incorporated. In certain embodiments, the surface treatment enhances the spreadability of the cosmetic composition. In certain embodiments, the surface treatment enhances the adhesion, water repellence, sebum absorption and/or surface smoothness of the cosmetic composition, for example, when applied to human skin.

In certain embodiments, the talc particulate is surface treated to increase the hydrophobicity or lipophilicity of the talc particulate.

In certain embodiments, the talc particulate is surface treated to increase the hydrophilicity or lipophobicity of the talc particulate.

In certain embodiments, the surface treatment is an organosilane, a organophosphour, an organosulfur, or a mixture thereof.

In certain embodiments, the surface treatment agent is a hydrocarbyl phosphonic acid, for example, an alkyl phosphonic acid such as, for example, a C₆-C₂₄ phosphonic acid, for example, n-octadecylphosphonic acid. In certain embodiments, the surface treatment agent is a halo-alkyl phosphonic acid such as, for example, a fluoro-alkyl phosphonic acid such as, for example, nonafluoropentadecyl phosphonic acid.

In certain embodiments, the surface treatment is selected from one or more methicone, diemthicone, triethoxysilane, lauroyl lysine, C₉₋₁₅ flouroalcohol phosphates, magnesium myristate, triethoxy caprylsilane, polyhdyroxystearic acid and perflourooctyl triethoxysilane,

In certain embodiments, the surface treatment is or comprises microcrystalline cellulose.

In certain embodiments, the surface treatment is or comprises polyacrylate.

In certain embodiments, the surface treatment is or comprises a polyoxyalkylene (POA), for example, polyalkylene glycol (PAG) or polyalkylene oxide (PAO). As used herein, the term ‘polyalkylene glycol’ means a POA having a number average molecular mass below 20,000 g/mol, and the term ‘polyalkylene oxide’ means a POA having a number average molecular mass above 20,000 g/mol. In certain embodiments, the surface treatment comprises or is a polyalkylene glycol having a number average molecular mass of from about 100 to about 15,000 g/mo, for example, from about 200 to about 10,000 g/mol, or from about 500 to about 9000 g/mol, or from about 1000 to about 9000 g/mol, or from about 2000 to about 900 g/mol, or from about 4000 to about 9000 g/mol, or from about 6000 to about 9000 g/mol, or from about 6000 to about 8500 g/mol.

In certain embodiments, the surface treatment is or comprises a polyalkylene oxide selected from one or more of paraformaldehyde (polymethylene oxide), polytetramethylene glycol, polytetramehtylene ether glycol, polyethylene oxide, polypropylene oxide, polybutylene oxide, and combinations thereof.

In certain embodiments, the surface treatment is or comprises a polyethylene glycol. In certain embodiments, the surface treatment comprises or is a mixture of polyethylene glycol and polypropylene glycol (PPG).

In certain embodiments, the surface treatment is or comprises a polyether modified polysiloxane. The polyether modified polysiloxane may be derived from a linear polysiloxane. In certain embodiments, the polyether modified polysiloxane is derived from poly(dimethylsiloxane), poly(hexamethyldisiloxane), poly(octamethyltrisiloxane), poly(decamethyltetrasilozne), or combinations thereof.

In certain embodiments, the surface treatment comprises at least one siloxane. In general, siloxanes are any of a class of organic or inorganic chemical compounds comprising silicon, oxygen, and often carbon and hydrogen, based on the general empirical formula of R₂SiO, where R may be an alkyl group. Exemplary siloxanes include, but are not limited to, dimethylsiloxane, methylphenylsiloxane, methylhydrogen siloxane, methylhydrogen polysiloxane, methyltrimethoxysilane, octamethylcyclotetrasiloxane, hexamethyldisiloxane, diphenylsiloxane, and copolymers or blends of copolymers of any combination of monophenylsiloxaneunits, diphenylsiloxane units, phenylmethylsiloxane units, dimethylsiloxane units, monomethylsiloxane units, vinylsiloxane units, phenylvinylsiloxane units, methylvinylsiloxane units, ethylsiloxane units, phenylethylsiloxane units, ethylmethylsiloxane units, ethylvinylsiloxane units, or diethylsiloxane units.

In certain embodiments, the surface treatment comprises an amine, or an amine derivative. In certain embodiments, the surface treatment comprises an alkylated amine, for example, an alkylated alkyl amine such, as for example, an ethylated alkyl amine. In certain embodiments, the surface treatment comprises an alkoxylated amine, for example, an ethoxylated amine, or an alkoylated alkyl amine, such as, for example, an ethoxylated alkyl amine.

In certain embodiments, the surface treatment comprises a polyalkylene glycol (PAG) and amine, for example, a PAG, an alkoxylated amine and a siloxane. In certain embodiments, the surface treatment comprises PAG (e.g., PEG), ethoxylated alkyl amine a siloxane. In certain embodiments, the surface treatment consists essentially or consists of the aforementioned surface treatment agents.

The amount of surface treatment may be any suitable amount, for example, an amount suitable to enhance one or more of the properties described above, e.g., spreadability, adhesion, water repellence, sebum absorption and/or surface smoothness. In certain embodiments, the surface treatment is present in an amount up to about 5 wt. %, based on the total weight of talc particulate, for example, from about 0.001 wt. % to about 5 wt. %, or from about 0.01 wt. % to about 2 wt. %, or from about 0.1 wt. % to about 2 wt. %, or from about 0.5 wt. % to about 1.5 wt. %, based on the total weight of talc particulate.

### EXAMPLES

The talc particulates described in Table 1 below were used to prepare a series of pressed powder compacts, as described in more detail in the examples below.

TABLE 1

| | Comparative | Comparative | | | | |
|---|---|---|---|---|---|---|
| | Talc A | Talc B | Talc 1 | Talc 2 | Talc 3 | Talc 4 |
| d_{50sedi} | 3.4 | 13.4 | 2.9 | 3.3 | 13.6 | 1.9 |
| d₅₀ₗₐₛₑᵣ | 8.4 | 22.7 | 23.7 | 20.0 | 34.0 | 9.8 |
| Lamellarity | 1.5 | 0.7 | 7.2 | 5.1 | 1.5 | 4.2 |
| index | | | | | | |
| BET (m²/g) | 6.5 | 3.0 | 22.0 | 22.8 | 5.4 | 19.3 |
| Aluminium | 0.6 | 0.6 | 0.6 | 0.6 | 0.6 | 12 |
| content (%) | | | | | | |

### Example 1

Three powder compacts were prepared as detailed in Table 2. The colour boosting attributes of each talc was determined by measuring the density of colour with a spectrocalorimeter according to ISO 12647. Results are given in Table 2. A ‘+’ indicates an increase in colour density, i.e., a colour boost, whereas a ‘-’ indicates a detrimental effect on colour density.

TABLE 2

| | Comparative | | |
|---|---|---|---|
| | compact | Compact 1 | Compact 2 |
| Comparative Talc A (wt. %) | 80 | 0 | 0 |
| Talc 1 (wt. %) | 0 | 80 | 0 |
| Talc 3 (wt. %) | 0 | 0 | 80 |
| Ultramarine pigment (wt. %) | 10 | 10 | 10 |
| Binder (wt. %) | 10 | 10 | 10 |
| Density of colour (+/−) | − | + | + |

As can be seen, Talc 1 and Talc 3 boost the colour of the compact.

### Example 2

The cohesion properties of Comparative Talc A, Comparative Talc B and Talc 1 from example 1 were each tested.

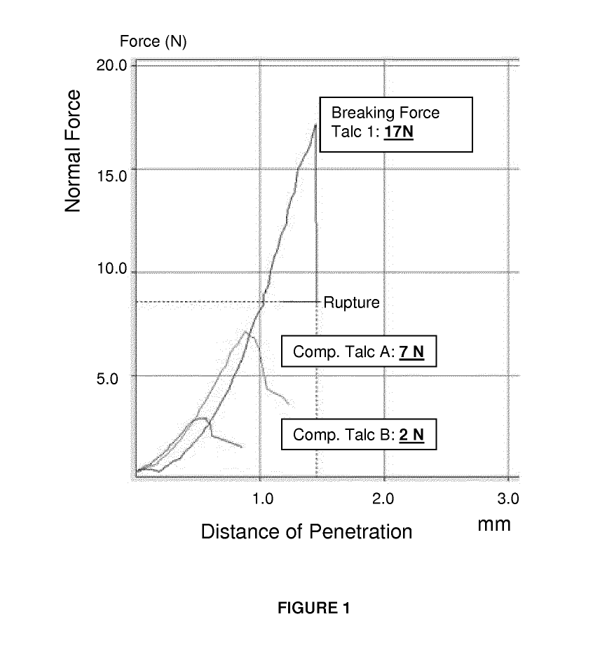

Cohesion was determined by measuring the fracture resistance of each talc in pressed tablet form. The tablet (diameter=57 mm; thickness=3 mm) was formed by pressing at 1500 psi. During the test, a ball probe (4 mm in diameter) is moved down onto an upper surface of the pressed tablet, and the normal force (stress) during penetration of the ball probe into the tablet is measured (using a texture analyzer). The loading rate is 100 mm/min. The test is continued until the tablet breaks. The Breaking Force (N) is the force measured when the table breaks. Results are presented in FIG. 1. As can be seen, Talc 1 has high cohesion in pressed tablet form, which enables process improvement and better handling.

### Example 3

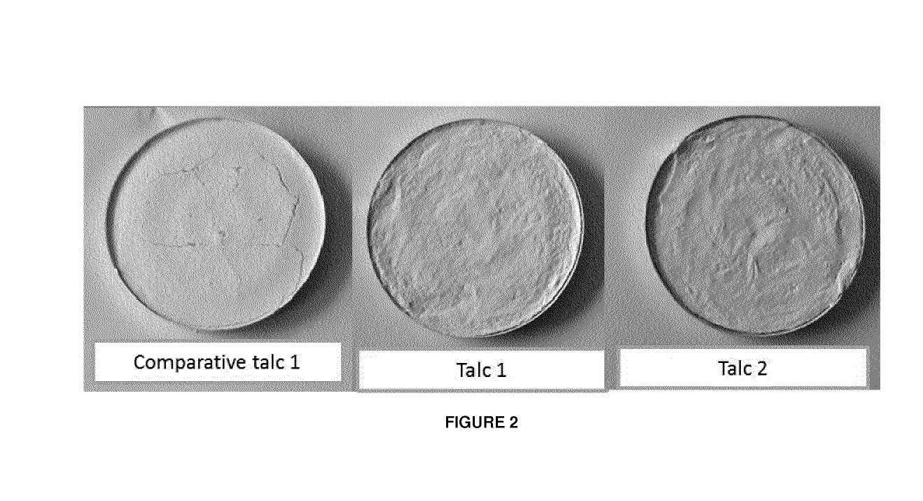

Compacts were prepared by a wet processing method using Comparative Talc A, Talc 1 and Talc 2. Slurries were formed with each talc, prepared at the same viscosity, placed in moulds and then dried at 80° C. Processing and drying conditions were identical for each compact. Following drying, a photograph of each compact was taken, as presented in FIG. 2. As can been seen, compacts made from Talc 1 and Talc 2 are visually free of cracks.

### Example 4

Powder formulations (83 wt. % talc particulate plus comparable amounts of pigment and binder) were prepared using Talc B, Talc 1 and Talc 4. The coverage and opacity of the powder formulations were assessed as follows.

Coverage—panel test (10 panellists) in which panellists apply powders to face and asked to rank them in terms of coverage categories: excellent coverage, very good coverage, acceptable coverage, and poor coverage.

Opacity (%)—the powder formulation is applied to black fabric with a cosmetic brush. Light reflectance is measured and opacity calculated.

Results are summarized in Tables 3 and 4.

TABLE 3

| | TALC 1 | TALC 4 | TALC B |
|---|---|---|---|
| Excellent Coverage | 0% | 25% | 0% |
| Very Good Coverage | 25% | 35% | 17% |
| Acceptable Coverage | 33% | 33% | 42% |
| Poor Coverage | 42% | 7% | 41% |

TABLE 4

| | TALC 1 | TALC 4 | TALC B |
|---|---|---|---|
| Opacity (%) | 76 | 84 | 62 |

For the avoidance of doubt, the present application extends to the subject-matter in the following numbered paragraphs (1 to 20):
1. Cosmetic composition comprising colourant and an inorganic particulate material having a d₅₀ₗₐₛₑᵣ of at least about 5.0 μm, for example, at least about 10.0 μm, a lamellarity index of at least about 1.0, and wherein the inorganic particulate material particulate comprises less than about 10% aluminium, based on the weight of the inorganic particulate material.
2. A cosmetic composition for application to human skin, said cosmetic composition comprising colourant and a colour boosting amount of an inorganic particulate material, wherein the inorganic particulate material boosts the colour of the cosmetic composition (i) such that a lower amount of the cosmetic composition may be applied to any given surface area of the skin without loss of colour, and/or (ii) such that the colour density of the cosmetic composition is boosted compared to the cosmetic composition absent the inorganic particulate material, and/or (iii) a lower amount of colourant may be used without loss of colour density compared to the cosmetic composition absent the inorganic particulate material.
3. Cosmetic method for modifying the visual appearance of a facial feature, comprising applying, to the facial feature, a cosmetic composition comprising colourant and a colour boosting amount of an inorganic particulate material.
4. Cosmetic method according to paragraph 3, wherein the presence of the colour boosting amount of the inorganic particulate material boosts the colour such that (i) a lower amount of the cosmetic composition may be applied to any given surface area of the facial feature without loss of colour, and/or (ii) a lower amount of colourant may be used without loss of colour density.
5. Cosmetic method according to paragraph 3 or 4, wherein the facial feature is one or more of skin, lips, eyebrow or eyelash.
6. Use as a colour booster in a cosmetic composition of an inorganic particulate material.
7. Use according to paragraph 6, wherein the inorganic particulate material has a d₅₀ₗₐₛₑᵣ of at least about 5.0 μm, for example, at least about 10.0 μm, and a lamellarity index of at least about 1.0.
8. A pressed powder cosmetic comprising a cohesion enhancer, said cohesion enhancer comprising, consisting essentially of, or consisting of, an inorganic particulate material having a d₅₀ₗₐₛₑᵣ of at least about 5.0 μm, for example, at least about 10.0 μm, and a lamellarity index of at least about 1.0, and wherein the inorganic particulate material comprises less than about 10% aluminium, based on the weight of the inorganic particulate material.
9. Use as a cohesion enhancer in a pressed powder cosmetic of an inorganic particulate material.
10. Use according to paragraph 9, wherein the inorganic particulate material has a d₅₀ₗₐₛₑᵣ of at least about 5.0 μm, for example, at least about 10.0 μm, and a lamellarity index of at least about 1.0.
11. Use according to paragraph 9 or 10, wherein an increase in cohesion is determined by measuring the fracture resistance of the pressed powdered cosmetic in tablet form, and wherein the increase in cohesion is determined relative to a comparable powdered cosmetic compact comprising the same amount of an inorganic particulate material which does not have a d₅₀ₗₐₛₑᵣ of at least about 5.0 μm, for example, at least about 10.0 μm, and a lamellarity index of at least about 1.0.
12. A method for enhancing the cohesion of a powdered cosmetic compact, the method comprising incorporating an inorganic particulate material having a d₅₀ₗₐₛₑᵣ of at least about 5.0 μm, for example, at least about 10.0 μm, and a lamellarity index of at least about 1.0 during manufacture of the powdered cosmetic compact.
13. A pressed powder cosmetic comprising a crack prevention additive, said crack prevention additive comprising, consisting essentially of, or consisting of, an inorganic particulate material having a d₅₀ₗₐₛₑᵣ of at least about 5.0 μm, for example, at least about 10.0 μm, and a lamellarity index of at least about 1.0, and wherein the inorganic particulate material comprises less than about 10% aluminium, based on the weight of the inorganic particulate material.
14. Use as a crack prevention additive in a pressed powder cosmetic of an inorganic particulate material.
15. Use according to paragraph 14, wherein the inorganic particulate material is used in slurry form, wherein the powdered cosmetic compact is manufactured by a wet pressing method, and wherein: (i) the inorganic particulate material has d₅₀ₗₐₛₑᵣ of at least about 5.0 μm, for example, at least about 10.0 μm, and a lamellarity index of at least about 1.0, and/or (ii) the inorganic particulate material is uncoated, and/or (iii) the pressed powder cosmetic comprises an oil-based binder.
16. A method for preventing cracking of a pressed powder cosmetic, the method comprising incorporating an inorganic particulate material in slurry form in a pressed powder cosmetic compact precursor, and wet processing said precursor to produce a pressed powder cosmetic which is visually free of cracks, optionally wherein (i) the inorganic particulate material has d₅₀ₗₐₛₑᵣ of at least about 5.0 μm, for example, at least about 10.0 μm, and a lamellarity index of at least about 1.0, and/or (ii) the inorganic particulate material is uncoated, and/or (iii) the pressed powder cosmetic comprises an oil-based binder.
17. Use as a colour booster, cohesion enhancer and crack prevention additive in a pressed powder cosmetic of an inorganic particulate material, optionally wherein the inorganic particulate material has d₅₀ₗₐₛₑᵣ of at least about 5.0 μm, for example, at least about 10.0 μm, and a lamellarity index of at least about 1.0.
18. Cosmetic composition, or pressed powder cosmetic, or method, or use according to any preceding paragraph, wherein the inorganic particulate material has a lamellarity index of at least about 2.0, for example, at least about 4.0, or at least about 6.0.
19. Cosmetic composition, or pressed powder cosmetic, or method, or use according to any preceding paragraph, wherein the inorganic particulate material has an aluminium content of no greater than about 5%, for example, no greater than about 2%, for example, no greater than about 1%.
20. Cosmetic composition, or pressed powder cosmetic, or method, or use according to any preceding paragraph, wherein the inorganic particulate material has:
   (i) a lamellarity index of from about 6.0 to about 8.0, for example, from about 6.5 to about 7.5, a d₅₀ₗₐₛₑᵣ of from about 20.0 μm to about 25.0 μm, and optionally a d_{50sedi} of from about 2.5 μm to about 3.5 μm; or
   (ii) a lamellarity index of from about 1.0 to about 3.0, for example, from about 1.0 to about 2.5, or from about 1.25 to about 2.0, a d₅₀ₗₐₛₑᵣ of from about 30.0 μm to about 40.0 μm, and optionally a d_{50sedi} of from about 10.0 μm to about 15.0 μm; or
   (iii) a lamellarity index of from about 4.0 to about 6.0, for example, from about 4.5 to about 5.5, a d₅₀ₗₐₛₑᵣ of from about 15.0 μm to about 25.0 μm, and optionally a d_{50sedi} of from about 3.0 μm to about 4.0 μm;
   (iv) a lamellarity index of from about 3.5 to about 5.0, for example, from about 4.0 to about 5.0, a a d₅₀ₗₐₛₑᵣ of from about 8.0 μm to about 12.0 μm, and optionally a d_{50sedi} of from about 1.5 μm to about 3.0 μm, and optionally an aluminium content of from about 10% to about 15%, based on the total weight of the talc particulate.

1. A cosmetic composition comprising a talc particulate having a d₅₀ₗₐₛₑᵣ of at least about 5.0 μm and a lamellarity index of at least about 1.0,
wherein the talc particulate comprises less than about 20% aluminium, based on the weight of the talc particulate.

2. A cosmetic composition for application to human skin, said cosmetic composition comprising
a colourant and
a colour boosting amount of a talc particulate,
wherein the talc particulate boosts the colour of the cosmetic composition
(i) such that a lower amount of the cosmetic composition may be applied to any given surface area of the skin without loss of colour, and/or
(ii) such that the colour density of the cosmetic composition is boosted compared to the cosmetic composition absent the talc particulate, and/or
(iii) a lower amount of colourant may be used without loss of colour density compared to the cosmetic composition absent the talc particulate.

3. A method for modifying the visual appearance of a facial feature, comprising applying, to the facial feature, a cosmetic composition comprising colourant and a colour boosting amount of a talc particulate.

4. A method according to claim 3, wherein the presence of the colour boosting amount of the talc particulate boosts the colour such that
(i) a lower amount of the cosmetic composition may be applied to any given surface area of the facial feature without loss of colour, and/or
(ii) a lower amount of colourant may be used without loss of colour density.

5. (canceled)

6. A cosmetic composition according to claim 1, wherein the cosmetic composition is a cohesion enhancer.

7. A pressed powder cosmetic comprising the cohesion enhancer of claim 6.

8. (canceled)

9. A powdered cosmetic compact comprising the cosmetic composition of claim 1.

10. A pressed powder cosmetic comprising a crack prevention additive, wherein said crack prevention additive comprises the cosmetic composition of claim 1.

11. (canceled)

12. The pressed powder cosmetic of claim 10, wherein the powdered cosmetic compact is manufactured by a wet pressing method, and wherein: the talc particulate is uncoated or the pressed powder cosmetic comprises an on-based binder.

13. A method for preventing cracking of a pressed powder cosmetic, the method comprising incorporating a talc particulate in slurry form in a pressed powder cosmetic compact precursor, and wet processing said precursor to produce a pressed powder cosmetic.

14. A pressed powder cosmetic comprising the cosmetic composition of claim 1, wherein the cosemic composition is a colour booster, cohesion enhancer and crack prevention-additive.

15. A cosmetic composition comprising a coverage and/or opacity enhancer, wherein said coverage and/or opacity enhancer comprises the cosmetic composition of claim 1.

16. A cosmetic composition for application to human skin according to claim 2, wherein the talc particulate has a d₅₀ₗₐₛₑᵣ of at least about 5.0 μm, a lamellarity index of at least about 1.0, and an aluminium content of less than about 20%, based on the weight of the talc particulate.

17. A cosmetic composition for application to human skin according to claim 16, wherein the cosmetic composition is a foundation or concealer.

18. (canceled)

19. A cosmetic composition for application to human skin according to claim 16, wherein the coverage and/or opacity enhancer is present in amount sufficient to provide: (i) full coverage when applied to human skin, and/or (ii) an opacity of at least about 70%.

20. A cosmetic composition according to claim 1, wherein the talc particulate has a d₅₀ₗₐₛₑᵣ of at least about 10.0 μm, a lamellarity index of at least about 1.0 and an aluminium content of less than about 10%, based on the weight of the talc particulate.

21. A cosmetic composition according to claim 1, wherein (i) the talc particulate has a lamellarity index of at least about 2.0, and/or (ii) the talc particulate has an aluminium content of no greater than about 5%.

22. A cosmetic composition according to claim 1, wherein the talc particulate is surface treated.

23. (canceled)

24. (canceled)

25. A cosmetic composition according to claim 20, wherein wherein the talc particulate is a surface treated talc particulate.

26. A cosmetic composition according to claim 25, wherein the talc particulate has a d₅₀ₗₐₛₑᵣ of at least about 10.0 μm and wherein the talc particulate comprises less than about 10% aluminium, based on the weight of the talc particulate.

27. A cosmetic composition according to claim 1, further comprising a colourant.
